(19) United States
(12) Patent Application Publication
(10) Pub. No.: US 2002/0068296 A1
(43) Pub. Date: Jun. 6, 2002

(54) Cathodic protection of nucleic acid sequences

(76) Inventor: Adam Heller, Austin, TX (US)

Correspondence Address: MERCHANT & GOULD PC, P.O. BOX 2903, MINNEAPOLIS, MN, 55402-0903, US

(21) Appl. No.: 09/907,279

(22) Filed: Jul. 17, 2001

Related U.S. Application Data
(60) Provisional application No. 60/218,959, filed on Jul. 17, 2000.

(51) Int. Cl. C12Q 1/68; C07H 21/04; C09K 3/00; C12N 5/06

(52) U.S. Cl. 435/6; 536/23.1; 435/325; 252/380

(57) ABSTRACT

Cathodic protection of a target polynucleotide molecule against oxidative damage is conferred by providing the target polynucleotide molecule with a protective, reducing agent positioned proximal to the nucleic acid sequence.

This application claims the benefit of U.S. Provisional Application Ser. No. 60/218,959 filed Jul. 17, 2000, the disclosure of which is incorporated in its entirety.

### FIELD OF THE INVENTION

The present invention relates to protection of nucleic acid sequences from oxidative damage. More specifically, the invention relates to conducting or semi-conducting nucleic acid sequences containing at least one protective reducing agent that functions as an oxidizable center, protecting a desired nucleic acid sequence from oxidative damage.

### BACKGROUND OF THE INVENTION

The intracellular oxidative state has recently gained much attention as playing a significant role in the functioning and condition of the cell. Changes in the intracellular oxidative state brought about by environmental factors such as electromagnetic radiation and chemical compounds are thought to play a role in the etiology of apoptosis, neoplastic transformation, carcinogenesis, and other cellular abnormalities associated with cellular proliferative disorders. For example, see Stohs, 1995, J Basic Clin Physiol Pharmacol; 6:205-28; Marnett, 2000, Carcinogenesis; 21:361-70; Nose, 2000, Biol Pharm Bull; 23:897-903. Oxidative stress has also been associated with various disease states, such as Alzheimer's Disease (Butterfield, et al., 2001, Curr Med Chem; 8:815-28) and atherosclerosis (Bennett, 2001, Circ Res; 88:648-50). An underlying molecular basis for cellular oxidative damage and its impact on the physiological state of the cell appears to be the intracellular formation of reactive radical species and the alteration of various cellular components caused by reaction with these species.

In response to various oxidizing factors, such as UV irradiation and chemical mutagens, cells can develop various reactive radical species, such as reactive nitrogen species, sulfate radicals, nitrogen-centered radicals, oxygen radicals, and lipid radicals. These compounds, as well as other reactive radical species, can chemically alter cellular components such as nucleic acids or proteins. Increased intracellular radical species can also change the concentration or reactivity of intracellular second messengers thereby modulating signal transduction pathways. These modulations can potentially affect cell growth or differentiation.

Of particular interest has been the effect of oxidizing radicals on genetic components in the cell. DNA damage can occur from intermediates of oxygen reduction that either attack the bases or the deoxyribosyl backbone of DNA. Oxygen radicals can also attack other cellular components, such as lipids, to generate reactive intermediate compounds that couple to DNA bases. For example, DNA is damaged by benzoyloxyl radicals that specifically cause damage to the 5′-G in a GG sequence (Kawanishi, et al., 1999, Biochemistry; 38:16733-9). UVA radiation also causes DNA damage at this site through electron transfer in the presence of certain photosensitizers (Kidd, et al., 2000, J Photochem Photobiol B; 58:26-31). Oxidative damage to various genes involved in cellular proliferation, for example oncogenes and tumor suppressor genes, has the potential to promote unregulated cellular proliferation resulting in tumor formation or the occurrence of cancer. Accordingly, compounds, particularly anti-oxidants, that prevent the damaging effects of reactive radicals on cellular components are needed to prevent such oxidative cellular damage.

Anti-oxidants, are generally thought to play a role in preventing oxidative damage caused by environmental factors. Physical consumption and application of anti-oxidants has wide public acceptance. Compounds with anti-oxidant properties, such as selenium, Vitamin D, Vitamin E, and Vitamin C, and carotenoids are available to the public and are commonly found in oral and topical formulations. Many studies have focused on these compounds, particularly in relation to purported protective effects on DNA, and some have investigated the molecular basis for these protective effects (see, for example, El-Bayoumy, 2001, Mutat Res; 475:123-39; Hartwig, 2001, Mutat Res; 475:113-21; Chatterjee, 2001, Mutat Res; 475:69-87; Claycombe and Meydani, 2001, Mutat Res; 475:37-44; Halliwell, 2001, Mutat Res; 475:29-35; Collins, 2001, Mutat Res; 475:21-8). Despite this research, the relationship between administration of antioxidants and protection against oxidative DNA damage in cells is not well understood.

There is a need for novel approaches and methods for preventing oxidative damage to cellular components, in particular to cellular nucleic acid molecules. The current invention provides methods and reagents that reduce or prevent oxidative damage to nucleic acid molecules.

### SUMMARY OF THE INVENTION

The current invention generally provides methods and reagents useful for the protection of nucleic acid molecules (polynucleotides) from oxidative damage. More specifically, the invention provides a reducing agent in a position that is electrically “downstream” or “proximal” to a target polynucleotide, where the reducing compound is in electrical communication with the target polynucleotide. In the presence of an oxidative event on the target polynucleotide molecule, the polynucleotide is able to electrically transmit a “hole”, resulting from the attack of the oxidizing agent, from the target polynucleotide molecule to the reducing agent, which is sacrificially oxidized.

A reducing agent useful in the current invention can be a G-rich polynucleotide sequence (nucleic acid sequence containing a high molar content of deoxyguanylate residues), a non-G reducing compound, a reducing protein, or a combination of these. In the method invention, the reducing agent can be positioned proximal to the target polynucleotide molecule, for example, in a fusion polynucleotide molecule that contains both the target polynucleotide molecule and the reducing agent, for example, a G-rich sequence. Alternatively, the reducing agent can be attached to a linker oligonucleotide that hybridizes to a nucleic acid sequence proximal to the target polynucleotide molecule.

The reducing agent, for example, a non-G reducing compound, can be positioned proximal to the target polynucleotide molecule by coupling the reducing compound to a nucleic acid that is proximal to the target polynucleotide molecule. A spacer moiety can be used to accomplish this coupling. A non-G reducing compound can also be positioned proximal to the target polynucleotide molecule via a linker oligonucleotide that is coupled to the non-G reducing compound and can hybridize proximal to the target polynucleotide molecule.

A reducing protein can be positioned proximal to the target polynucleotide molecule by binding the reducing protein to a protein-binding DNA sequence positioned proximal to the target polynucleotide molecule. The reducing protein can be, for example, a chimeric protein that contains a reducing portion and a DNA binding portion. A linker oligonucleotide that contains a protein-binding DNA sequence to can also be used to bind the reducing protein. The linker oligonucleotide can hybridize proximal to the nucleic acids.

The target polynucleotide molecule can be a DNA or RNA.

The invention also provides useful materials that contain reducing agents, for example oligonucleotides and vectors, that can be used for cathodic protection of a target polynucleotide molecule against oxidative damage. These materials are useful in various applications including, but not limited to, polymerase chain reaction methods (PCR), genetic engineering methods, production of proteins from cells culture, and the like.

### BRIEF DESCRIPTION OF THE FIGURES

FIG. 1 is a computer-generated ball model showing possible channels in which protons may shift concertedly in an electric field paralleling the main axis of a double stranded nucleic acid sequence.

FIG. 2 is a stick model of the molecule shown in FIG. 1.

FIG. 3 is a magnified portion of the stick model of FIG. 2.

FIG. 4A is a schematic representation of a non-conducting rod-shaped macromolecule under attack by an oxidizing agent and FIG. 4B is a schematic representation of a localized radical generated following attack by the oxidizing agent.

FIG. 5A-5D are schematic representations of cathodic protection of a conducting rod-shaped macromolecule via sacrificial oxidation of a reducing agent. FIG. 5A schematically illustrates target polynucleotide molecule attack by an OH radical; FIG. 5B schematically illustrates electron capture by the OH radical and reduction to an OH⁻ anion leaving a mobile electron vacancy, or hole (h⁺) in the target polynucleotide molecule; FIG. 5C schematically illustrates hole diffusion and capture by the reducing agent (right side of rod-shaped molecule); and FIG. 5D schematically illustrates formation of a radical at the reducing agent.

FIG. 6A is a schematic diagram of a double-stranded DNA molecule including a target DNA sequence and an upstream reducing G-rich DNA sequence.

FIG. 6B is a schematic diagram of a double-stranded DNA molecule including a target DNA sequence and two reducing G-rich DNA sequences.

FIG. 6C is a schematic diagram of a double-stranded DNA molecule including a target DNA sequence that contains an intron and exons and a reducing G-rich DNA sequence positioned on the non-coding strand within the intron.

FIG. 7A is a schematic diagram of a double-stranded DNA molecule having a target DNA sequence and a non-G reducing compound coupled within the target DNA sequence on the non-coding strand.

FIG. 7B is a schematic diagram of a double-stranded DNA molecule having a target DNA sequence and a non-G reducing compound coupled proximal to the target DNA sequence and on the coding strand.

FIG. 8A is a schematic diagram of a double-stranded DNA molecule having a target DNA sequence and a reducing protein bound proximal to the target DNA sequence on the non-coding strand.

FIG. 8B is a schematic diagram of a double-stranded DNA molecule having a target DNA sequence and a reducing protein bound within an intron on the target DNA sequence and on the coding strand.

FIG. 9 is a schematic diagram of a double-stranded DNA molecule having a target DNA sequence and a hybridized linker oligonucleotide that contains a hybridization sequence and a reducing G-rich sequence.

FIG. 10 is a schematic diagram of a double-stranded DNA molecule having a target DNA sequence and a hybridized linker oligonucleotide that is coupled to a non-G reducing compound.

FIG. 11 is a schematic diagram of a double-stranded DNA molecule having a target DNA sequence, a hybridized linker oligonucleotide that contains a hybridization sequence and a reducing protein binding sequence and a reducing protein bound to the reducing protein binding sequence.

FIG. 12 is a schematic diagram of an RNA molecule hybridized to a linker oligonucleotide having a reducing sequence.

FIG. 13 is a schematic diagram of an RNA molecule hybridized to a linker oligonucleotide having a non-G reducing compound coupled to it.

FIG. 14 is a schematic diagram of an RNA molecule hybridized to a linker oligonucleotide having a reducing protein binding sequence and a reducing protein bound to the reducing protein binding sequence.

FIG. 15A is a schematic representation of the 12 base PQQ-bound double-stranded oligonucleotide (3′-ACGAAGGCTGAT-5′) hybrid on gold, Au—S—(CH₂)₂-double stranded oligo-NH—PQQ.

FIG. 15B is a schematic drawing of Au—S—(CH₂)₂—NH—PQQ, having no nucleotide attached.

FIG. 16A and 16B are graphic representations of cyclic voltammograms.

FIG. 16C is a graph showing the dependence of the peak separation Eₚ^{a}-E_{O} on the scan rate for monolayers on gold.

### DETAILED DESCRIPTION OF THE INVENTION

Definitions

As used herein, “target polynucleotide molecule” refers to a nucleic acid molecule, such as a DNA or RNA molecule, an RNA/DNA hybrid molecule, or a modified nucleic acid coupled or bound to another molecule that is protected from oxidative damage. The target polynucleotide molecule is typically a gene or a transcribed nucleic acid sequence. The target polynucleotide molecule can also include nucleic acid sequences that control the transcription of the gene.

As used herein, “transcribed region” refers to a portion of the target polynucleotide molecule that is transcribed into RNA by a RNA polymerase.

As used herein, “coding strand” or “template strand” refers to the strand of DNA from which RNA polymerase reads and polymerizes a RNA transcript from whereas “non-coding strand” or “non-template strand” refers to the strand that is complimentary to the coding strand.

As used herein, “oxidative damage” refers to chemical changes in deoxyribonucleic and ribonucleic acids as a consequence of being exposed to an oxidative agent. Oxidized DNA bases can include thymine glycol; 8-oxodeoxyguanosine; 5 hydoxymethyluracil; 6-hydroxy-5,6-dihydrocytosine; 5-hydroxyuracil; uracil glycol etheno- and propane-deoxynucleotide adducts. As used herein “oxidizing agent” refers to compounds that can cause chemical modifications to nucleic acid; these include hydroxy radicals (HO•), hydrogen peroxide (H₂O₂), peroxynitrite (ONO₂⁻), peroxynitrous acid (ONO₂H), and lipid peroxides such as malondialydehyde and 4-hydroxynonenal.

As used herein, “positioned” and “positioning” refers to the process of bringing a reducing agent into proximity with the target polynucleotide molecule to establish electrical contact between them. A reducing agent is positioned proximal to a target polynucleotide molecule if the reducing agent is on the same DNA strand or the complimentary DNA strand as the target polynucleotide molecule; if the reducing agent is bound (bound, for example, by covalent, ionic, coordinative, van der Waals binding, or hydrogen bonding interactions) to the same DNA strand or the complimentary DNA strand as the target polynucleotide molecule; if the reducing agent is bound to a oligonucleotide which is hybridized proximal to a target polynucleotide molecule; or combinations of these.

As used herein, “proximal” refers to a distance between the reducing agent and the target polynucleotide molecule where there is electrical contact between them, and where the electrical signal is transmitted away from the target polynucleotide molecule and toward the reducing agent.

As used herein “reducing agent” refers to a compound that has a lower reduction potential compared to the target polynucleotide molecule that it is proximal to. The reducing agent can be a G-rich nucleic acid segment, a non-G reducing compound, for example, a small molecule with a phenolic function, or a protein.

As used herein, “electrical communication” refers to the process of electrically delocalizing a charge or a hole from one or more points on a target polynucleotide molecule to one or more reducing agents located proximal to the target polynucleotide molecule.

As used herein, “linking” refers to the process of directly or indirectly coupling a reducing agent to a target polynucleotide molecule. “Linking agent” refers to a molecule that performs this coupling. Linking of the reducing agent to target polynucleotide molecule can include forming at least one covalent, ionic, coordinative, van der Waals, or hydrogen bond (or combinations of these bonds) between the reducing agent and target polynucleotide molecule. A “linker oligonucleotide” refers to a nucleic acid sequence that is able to hybridize to a sequence that is within or proximal to the target polynucleotide molecule and brings a reducing agent into proximity to the target polynucleotide molecule.

As used herein, “phenolic function” refers to an OH group bound to an aromatic hydrocarbon or to an aromatic heterocylic ring.

As used herein, “protected” refers to the effect of reducing or minimizing damage and destabilization of nucleic acid components due to oxidative agent attack.

Conductivity of Nucleic Acids

Various studies have shown the electrical conductivity of DNA sequences. In one study, the electrical diffusivity of DNA was measured with salmon testes DNA films cast on a slide (Okahata, et al., 1998, J Am Chem Soc, 120:6165). The DNA films were then reactively bound to interdigitated comb electrodes, spaced at micron distances, and a steady-state DC current was measured. The steady-state DC current measured resulted from electronic (non-ionic) conduction, by electrons or holes. The current was not from mobile cations and anions since they would have been exhausted from the inter-electrode space by migrating to their respective electrodes within about 1 minute (Aoki and Heller, 1993, J Phys Chem, 97:11014.)

Metals and intrinsic small band-gap semiconductors are black while DNA is colorless. The only known colorless conductors are highly doped large band-gap semiconductors. Among these, TiO₂ doped with hydrogen, with oxygen vacancies, or with Ti³⁺; similarly doped SrTiO₃; SnO₂ doped with indium or antimony; and In₂O₃ doped with tin, are particularly well known in electrochemistry, and can be applied as photoanodes in photoelectro-chemical cells and as transparent electrodes in spectro-electrochemical cells. The band gaps of all four semiconductors exceed 3 eV. These large band gap semiconductors conduct because their dielectric constants are high. Bohr-radii of donors or acceptors increase linearly with the high-frequency dielectric constant (Equation 1) (Kittel, Introduction to Solid State Physics, 5^{th} Edition, Wiley, 1976, p. 232). The radii, r_{O}, scale with the dielectrh²/(e²mₑ)(1) where ε is the dielectric constant is Heisenberg's constant, e is the charge of the electron, and mₑ is the effective electron mass. The Bohr radii are typically of 30-100 Å when the dielectric constants are between 10 and 20. The ionization energy of donors scale with the inverse of the square of the dielectric constant, (Kittel, Supra) according to Equation 2:

E_{g}−E_{d}=e⁴mₑ/(2ε²h²) (2)

When the high frequency dielectric constant is in the 10-20 range, the ionization energies shrink to tens of meVs, on the order of kT at ambient temperature. Thus the donors are ionized (Kittel, Supra).

Although the static dielectric constant of water is also high, there is a profound difference between water and DNA. The 7.4×10⁵ D theoretically estimated mean longitudinal thermal fluctuating dipole moment of DNA at 298° K. is 4×10⁵ times greater than the 1.84 D permanent dipole moment of water. Its estimated static longitudinal polarizability is 1.5×10⁻²⁷ F m⁻², or 9×10¹² times greater than that of water (Fomes, J. A., (1998), Phys Rev E; 57:2104). Therefore, the denser the non-randomly oriented DNA is, meaning the less water it contains, the higher its conductivity will be. The high conductivity allows the target polynucleotide molecule to be protected at great ranges by a reducing agent. When DNA is organized into densely packed aggregates, which are non-randomly oriented, the electrical conductivity will be high and the target polynucleotide molecule has the potential to be protected by a proximally positioned reducing agent.

The dielectric constant, ε, of DNA is unusually high (Takashima, 1973, Biopolymers, 12:145). At 100 kHz ε was estimated to be as high as 86 (Goswami, and Das Gupta, 1974, Biopolymers, 13:1549). G nucleotides are the most likely to be ionized since they are the easiest to oxidize of all the naturally occurring nucleotides (Brett, et al., 1994, J Electroanal Chem, 366:225; Tomschik, M., et al., 1999, J Electroanal Chem, 476:71). When not ionized, their orbitals in DNA resemble long sausages. When ionized, the electrons they donate are unidirectionally mobile at room temperature in the conduction band of DNA, making the aligned DNA duplexes conductive, doped or degenerate, semiconductors.

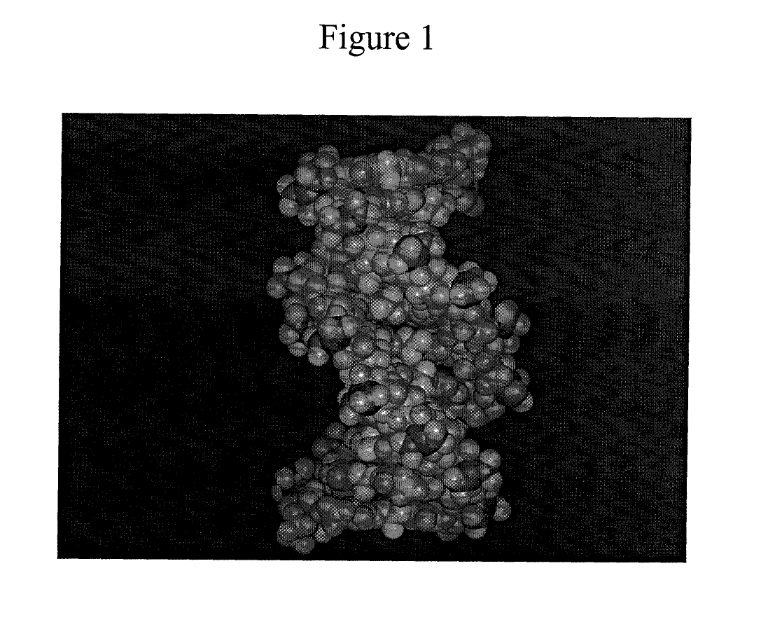

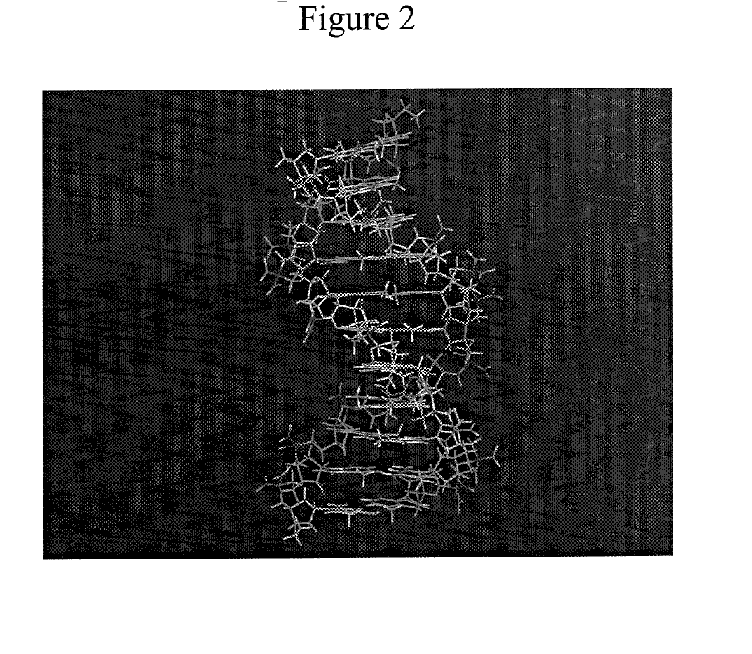

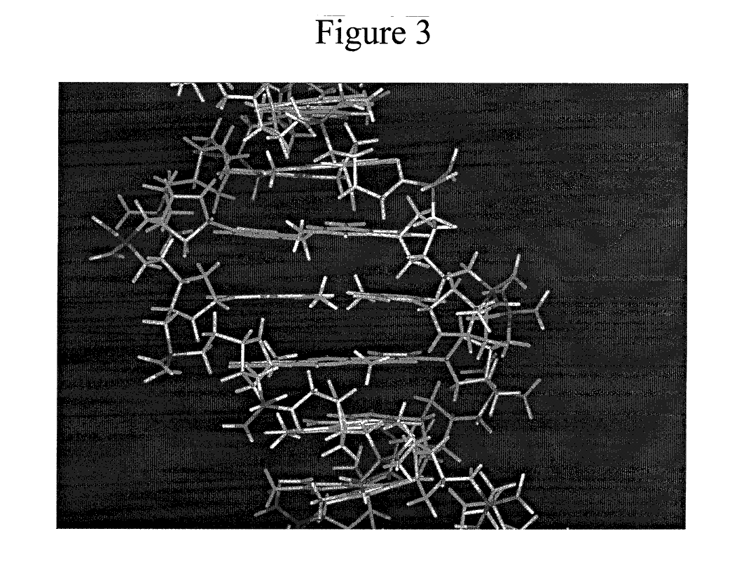

An array of parallel rod-like DNA duplexes can be polarized by protonation of one of their termini and ionization of their opposite termini. Concerted shift of protons or alkali metal cations between neighboring base-pairs can thus lead to polarization. FIGS. 1-3 show the existence of two channels through which protons can concertedly move. The phosphate channel spiraling on the outside of the DNA rods; and the amine channel, at the central axis of the rods, formed of the primary amines of G, C and A. The proton-transfer distances depend on the specific base pair sequences. The N-N distances between the primary amines of neighboring base-pairs are 4.2-5.5 Å. They are likely to be shorter for GC sequences than for AT sequences, because in GC both of the bases have primary amines, while in AT only A does. The O-O distances of neighboring phosphates are longer, 5.6-7.6 Å. Thus, the axial amine channel is more likely to define the polarizability than phosphate channel, and is therefore more likely to determine also the dielectric constant and the conductivity.

Because the protons shift concertedly in an electric field, the polarization times are short. Assuming that the diffusivity of protons, D, along the main axis is similar to that in water, about 10⁻⁴ cm²s⁻¹, then for the l≈4.2−5.5 Å amine spacings the inter-amine diffusion time, τ≈l²/D, is about 2×10⁻¹¹s.

High unidirectional polarizability, dielectric constant, and conductivity have been demonstrated for solid DNA arrays in which the molecules are non-randomly aligned. Preferably, the DNA is aligned so that at least parts of their sequences are about parallel. Electron conduction across 600 nm long DNA “ropes” (Fink, H. -W. and Schonenberger, C., 1999, Nature London, 398:407) and across macroscopic aligned DNA films (Lewis, D. et al.,1997, Science, 277:673) was reported only in solid DNA. The axial amine channels that enhance the polarizability and thus the transfer of electrons are found however in all DNA hybrids. Transfer of electrons across 10-20 base pairs (bp), for which the transfer-distance substantially exceeds the <2 nm maximal electron transfer distance in proteins, occurs in solutions. These distances are much shorter than the massively larger electron transfer distances required for the cathodic protection of genes, and do not approach the dimensions of folded chromosomes in chromatin. The spectroscopic and photochemical studies of DNA solutions establish, nevertheless, the principle of remote oxidation, and prove that the remote site oxidized is the 5′G of a poly-G sequence (see, for example, Hall, B. et al., 1996, Nature 382:731; Arkin, M. R. et al., 1997, Chem Biol 4:389; and Meggers, E., 1998, J Am Chem Soc, 120:12950).

Therefore, DNA helices are uniquely polarizable because protons can concertedly and rapidly shift (in less than 10⁻¹⁰ s) between the primary amines of their neighboring C, G and A bases and/or phosphates. The shift is faster and the polarizability is greater for GC sequences than for AT sequences because GC sequences have twice as many primary amines as AT sequences. The C, G and A-amines form a near-axial proton-transporting channel. Parallel alignment of the DNA helices increases their unidirectional bulk polarizability and dielectric constant. When the dielectric constant is high, the ionization energies of donor G-bases in DNA, viewed as a large band gap unidirectional semiconductor, approaches kT. As a result, the aligned DNA is an electronic conductor at room temperature.

Oxidative Damage of Nucleic Acids

The fidelity of the chromosomal information is at least, in part, maintained by excision and replacement of damaged segments (Croteau, D. L., and Bohr, V. A., 1997, J Biol Chem, 272:25409; Bohr, V. A., and Anson, R. M., 1995, Mutat Res, 338:25). Genes function, however, while exposed to oxidizers such as nitric oxide, singlet oxygen, hydrogen peroxide, and •OH radicals. The transient, approximately 1 nM, concentration of nitric oxide is similar to that of other hormones. There are transiently about 10⁶ nitric oxide molecules in a cell, any of which may react with the single copy of a particular chromosome or gene. Although catalases abound in tissues, some of the continuously generated hydrogen peroxide will react with oxidizable transition metal ions, such as Fe²⁺ or Cu⁺, to produce •OH radicals.

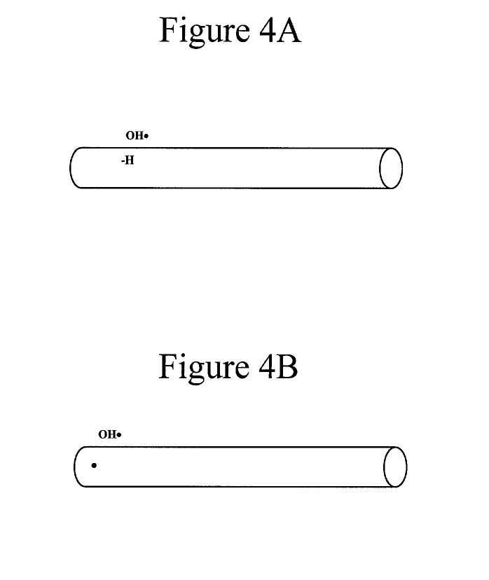

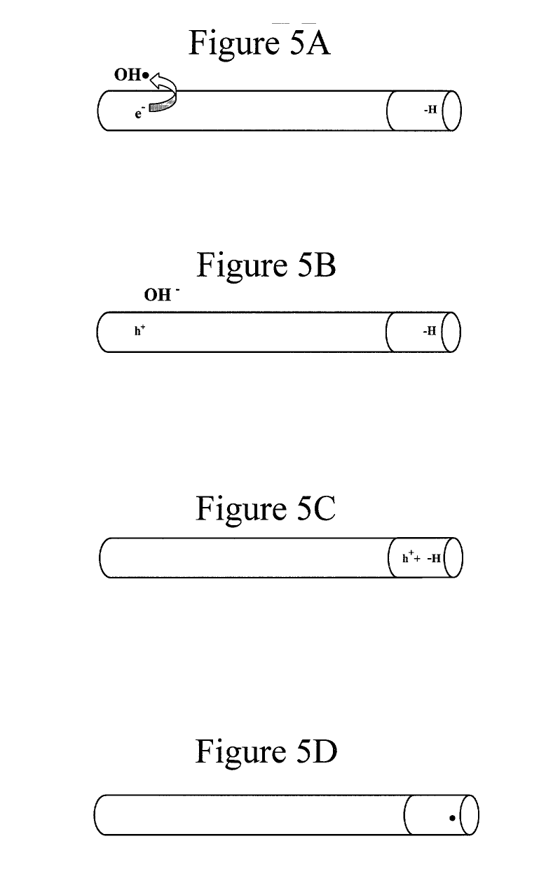

In an insulator, as illustrated in FIGS. 4A-4B, the attack by an oxidizer (4A) results in a local chemical change at the site of the attack (4B). In a conductor, as illustrated in FIG. 5A-5D, the reaction occurs at a remote reducing site to which the injected hole diffuses and in which it is trapped. Thus, there is a fundamental change in the oxidation of a target polynucleotide molecule if it undergoes an insulator-to-semiconductor transition. When in its insulating state, the site approached by the oxidizing agent is the site oxidized, as shown in FIG. 4A-4B. When in its conducting state, the site from which the electron is captured or into which the hole is injected is not the reaction site. Instead, as illustrated in FIG. 5B-5C in the conducting state the hole diffuses to, and reacts at, a region that includes a reducing agent. Finally, as illustrated in FIG. 5D, the reducing agent forms a radical species by the release of a proton, and essentially protects the target polynucleotide molecule from this type of damage.

“Cathodic” Protection of Nucleic Acids

The current invention generally describes methods and compositions useful for protecting a target polynucleotide molecule from oxidative damage by positioning a reducing agent proximal to the target polynucleotide molecule that is electrically conductive. The electrical conductivity of nucleic acids allows for the transport of a hole(s), caused by the reaction of an oxidant at a nucleotide along the target polynucleotide molecule to a reducing agent, positioned proximal to the target polynucleotide molecule, and which is more readily oxidized. The reducing agent is therefore sacrificed to protect the target polynucleotide molecule from the effects of the oxidant, a process herein referred to herein as “cathodic protection”.

According to the invention, the resistivity of the target polynucleotide molecule molecule is preferably less than about 1 Ω cm and more preferably less than about 10⁻³ Ω cm.

According to the invention, reducing agents are positioned proximal to a target polynucleotide molecule, thereby conferring protection against oxidative damage. Because oxidative damage to nucleic acid sequences is known to cause gene mutations, cell aging, and increased sensitivity to agents such as radiation and chemical agents, by protecting a target polynucleotide molecule(s) against such oxidative damage, the reducing agent provides protection of cells, tissues, and organisms against such cellular damage, aging, and increased sensitivity.

In addition, the invention also provides protection to target polynucleotide molecules for in vitro applications, for example PCR reactions, where an oxidizing environment may be present or can develop during the course of the reaction.

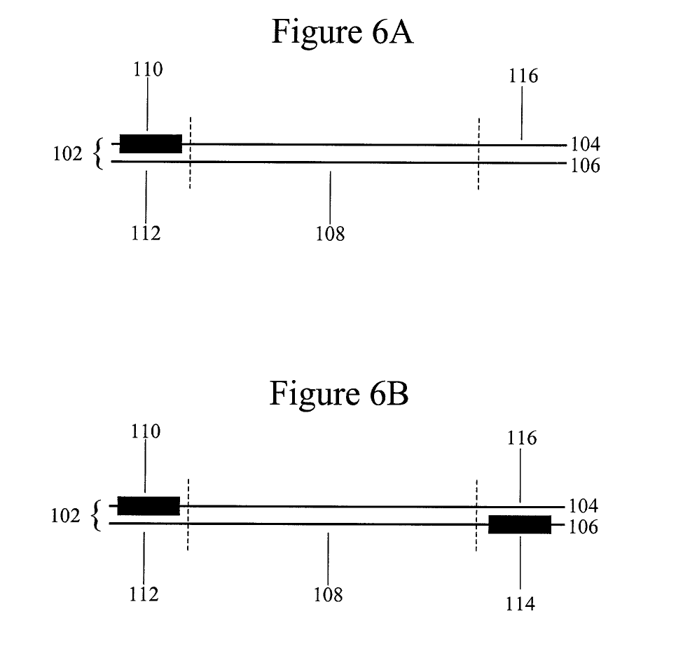

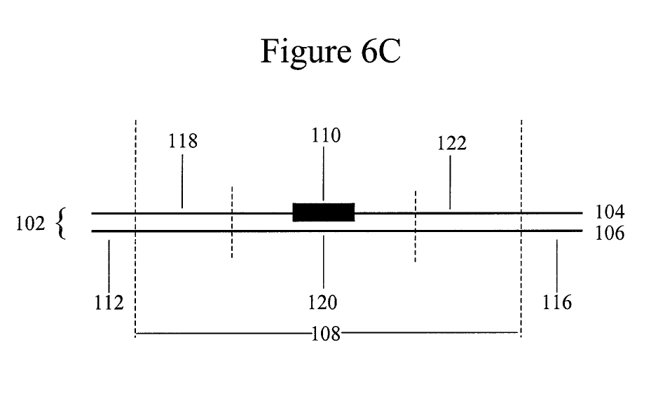

As illustrated in FIGS. 6A-6C, the invention includes a method of protecting a target polynucleotide molecule 108 from oxidative damage by positioning a reducing agent proximal to the polynucleotide molecule. The reducing agent can be, for example, a G-rich (deoxyguanylate-rich) sequence. As shown in FIG. 6A, the G-rich sequence 110 is positioned proximal to target polynucleotide molecule 108, for example being positioned within the first proximal nucleic acid region 112.

The target polynucleotide molecule 108 can be included in a double stranded DNA region 102 that also includes a first proximal nucleic acid region 112 and can also include second proximal nucleic acid region 116. Double stranded DNA region 102 is typically composed of first DNA strand 104 and second DNA strand 106 (See FIG. 6B). At any portion of the double stranded DNA sequence 102, first DNA strand 104 is complimentary to second DNA strand 106 and can hybridize by standard Watson-Crick base pairing.

For a target polynucleotide molecule 108 that is transcribed by an RNA polymerase, either first DNA strand 104 or second DNA strand 106 can serve as the template strand for transcriptional synthesis of a RNA molecule. Typically when one strand is the template strand the complimentary strand is the non-template strand.

The target polynucleotide molecule 108 contains nucleotides that are to be protected from chemical changes occurring from oxidative damage. A portion of the target polynucleotide molecule 108 can include a transcribed DNA sequence and typically this transcribed DNA sequence is a gene. A portion of the target polynucleotide molecule can be transcribed into an RNA molecule, for example, pre-messenger RNA (pre-mRNA), messenger RNA (mRNA), transfer RNA (tRNA), ribosomal RNA (rRNA), small nuclear RNA (snRNA), small nucleolar RNA (snoRNA), catalytically active RNA molecules, or other RNA molecules. The target polynucleotide molecule can also contain a transcribed region that includes exons and introns. Typically introns are removed during the process of RNA splicing in eukaryotic cells. The target polynucleotide molecule 108 can also include regulatory sequences that are involved in the regulation of transcription or overall gene expression of the target polynucleotide molecule 108. These regulatory sequences can include enhancers, promoters, repressors, initiators, and other nucleic acid sequences that either positively or negatively regulate the amount, timing, or cell-specific expression of the transcribed DNA of the target polynucleotide molecule 108. The target polynucleotide molecule 108 can also include various combinations of genes or portions of genes that encode, for example, chimeric proteins, and regulatory sequences controlling the expression of these genes. These combinations can be prepared by molecular biology techniques that are well established.

The G-rich sequence preferably contains a higher percentage of G residues per nucleotide based on a double stranded DNA molecule than present in an average nucleic acid sequence, for example, greater than 25%. More preferably, the G-rich sequences of the invention have a percentage of G in the range of 40-50%. The G-rich sequence is preferably 5 nucleotides or more in length, more preferably than 10 nucleotides or more in length, and most preferably 20 nucleotides or more in length. Preferably, the G-rich segment contains multiple G repeats, more preferably, two or more of GGG or GGGG repeats. The G-rich segment may include one or more of TTAGGG, GGGGTTGGGG, or GGGGTTTTGGGG. The G-rich sequence of the reducing agent 110 is more readily oxidized than the target polynucleotide molecule 108 and is preferably neutral or negatively charged at physiological pH.

A first G-rich sequence 110 can either be located on the non-template strand, for example, first strand 104, or the template strand, for example, second DNA strand 106. The G-rich sequence 110 is positioned proximal to the target polynucleotide molecule 108, preferably within a distance sufficient to permit efficient transport of electrons or holes along the double stranded DNA sequence 102 to the more reducing, G-rich sequences 110. Preferably, the distance between the most proximal ends of the G-rich sequence 110 and the target polynucleotide molecule 108, that is, for example, the 3′ end of the G-rich sequence 110 and the 5′ end of the target polynucleotide molecule 108, permits electrical conductivity between the reducing agent and the target polynucleotide molecule.

As illustrated in FIG. 6A, the first G-rich sequence 110 can be located on first DNA strand 104 within first proximal nucleic acid region 112; however the first G-rich sequence 110 can also be located within second proximal nucleic acid region 116. Alternatively, first G-rich sequence 110 can be located on second strand 106 either within first proximal nucleic acid region 112 or within second proximal nucleic acid region 116. This embodiment also includes, as illustrated in FIG. 6B, a double stranded DNA sequence 102 including target polynucleotide molecule 108, a first proximal nucleic acid region 112 including a first G-rich sequence 110 on first strand 104 and a second proximal nucleic acid region 116 including a second G-rich sequence 114 on second strand 106. In this example, the G-rich sequences are on different strands, however, these sequences can also be on the same strand.

The double stranded DNA region 102 that includes the target polynucleotide molecule 108, proximal nucleic acid region(s) 112 and 116, and G-rich sequence(s) 110 and 114 can be present in various in vivo or in vitro environments. The DNA region 102 can be present in vivo in a cell, for example, in a prokaryotic or eukaryotic cell. The cell or particle containing the DNA region 102 can be present in or a part of a multi-cellular organism. The DNA region 102 can be located, for example, within the chromosomal DNA in the nucleus or mitochondrial DNA. Alternatively, the DNA region 102 or can be episomal, for example, existing as a plasmid or vector inside the cell. Alternatively, the DNA region 102 can be included as part of viral DNA material, for example, either within a retroviral particle or within retroviral DNA that has been integrated into the genetic material of a cell which the retroviral particle has entered.

The double stranded DNA sequence 102 can be introduced into prokaryotic or eukaryotic cells by various methods. Typically the double stranded DNA 102 is included on a vector, such as a plasmid which can contain other genetic sequences, for example, selectable marker genes such as the beta-lactamase gene, or other genetic sequences that are involved in the maintenance or detection of the plasmid, such as a centrosomal sequence or a green fluorescence protein gene, respectively. Prokaryotic cells, for example, bacterial cells, can be transformed with DNA using methods such as, for example, electroporation or by using competent cells. DNA can be introduced into eukaryotic cells by methods such as, for example, lipofectamine transfection, calcium phosphate transfection, lithium acetate-polyethylene glycol transformation, or particle gun transformation. Techniques for introducing nucleic acids into cells are well known in the art and can be found in various references, such as Molecular Cloning: A Laboratory Manual (Ed.: Sambrook, J. and Russell, D. W., 3rd edition, Jan. 15, 2001, Cold Spring Harbor Laboratory Press, New York).

Cathodic protection of a target polynucleotide molecule 108 can be desirable in an oxidative environment. Cathodic protection can decrease the likelihood that the fidelity of target polynucleotide molecule 108 expression or the product of target polynucleotide molecule 108 expression is compromised. That is, oxidative damage to nucleotides of a particular gene can result in transcription defects, truncated protein products, or mutations in the protein products.

Cells that have been transformed or transfected with a vector containing the DNA sequence 102 including the target polynucleotide molecule 108 and reducing agent(s) 110/114 can be used to produce protein expressed from the target polynucleotide molecule 108. Expression of a cathodically protected gene is useful when the transformed cells are maintained and the target gene is expressed in oxidative conditions or when the environment changes during the maintenance or gene expression. For example, in eukaryotic cell culture, media conditions can change causing a pH change that is not controlled by the components of the media. It may be desirable that the protein of interest is cathodically protected from oxidative damage caused by changes in media conditions. Protein produced from a gene under cathodic protection is less likely to contain mutations resulting from oxidative damage. Protein produced and purified under cathodic protection can be useful in various applications, such as protein crystalography.

The double stranded DNA region 102 can also include nucleic acid sequences that allow for the mobility or integration of the DNA region 102 in other genetic material. As used herein “other genetic material” refers to nucleic acids, for example chromosomal DNA, plasmid DNA, or mitochondrial DNA, to which the double stranded DNA region 102 is targeted. Sequences that allow for mobility or integration include, for example, transposons, insertion sequences (IS elements), inverted repeats, and direct repeats. Preferably, these sequences encompass the DNA region 102 that includes the target polynucleotide molecule and the reducing agent and thereby allow integration of the target polynucleotide molecule and the reducing agent into DNA region 102 other genetic material. In addition, genes involved in mobility or integration, for example, transposases, integrases, recombinases, and resolvases can also be located on the DNA region 102.

The double stranded DNA region 102 can be introduced into cells and integrated into the hosts genetic material by various gene delivery systems, for example, by retroviral vectors, lentiviral vectors, adenoviral vectors, and adeno-associated viral vectors, virosomes (liposome/virus delivery), or MLV/VL30 chimeras (Donahue and Dunbar, 2001, Hum Gene Ther, 12:607-17; Solaiman, 2000, Mol Reprod Dev, 56:309-15). Integration of the double stranded DNA region 102 can also be achieved by via mobile Group II intron insertion and reverse-transcription (Guo, H. et al., 2000, Science, 289:452). For example, a RNA molecule containing the genetic information of double stranded DNA region 102, a mobile group II intron moiety, and a RNA moiety directing the insertion of the RNA molecule can be integrated into a desired location of the genetic material of a cell.

Another useful aspect of the current invention is the generation of transgenic mice containing a cathodically protected target polynucleotide molecule 108. These transgenic mice lines can be established, for example, by insertion of DNA 102 including a target polynucleotide molecule 108 and at least one reducing agent, for example, a G-rich sequence, positioned proximal to the target polynucleotide molecule 108, into the pronuclei of a fertilized mouse egg. The eggs can then be transferred to a foster female mouse and the offspring of these foster females can be tested for the integration of DNA into their chromosomes. Integration of the DNA 102 into the chromosome can be tested using various techniques, for example, by southern blotting. Alternatively, other genes can be present on the DNA 102 injected into the pronuclei of the fertilized mouse egg. These genes can be useful for detecting the integration of the DNA 102 into the chromosome.

The double-stranded DNA region 102 can be constructed using molecular biology techniques that are well known in the art and that can be found in various references, for example, in Current Protocols in Molecular Biology (Ed.: Ausubel et al., 1990, Greene Pub. Associates and Wiley-Interscience: John Wiley, New York). Sequences included in the double-stranded DNA region 102, for example, G-rich protective sequences, can be synthesized using commercial nucleic acid synthesizers (available from, for example, Applied Biosystems, Foster City, Calif.), or can be obtained commercially (for example, from Life Technologies, Rockville, Md.). It is understood that a vast combination of transcribed DNA sequences, regulatory elements, and G-rich sequences can be arranged or combined on the double stranded DNA region 102 through the use of molecular biology techniques.

In another example of this embodiment, as illustrated in FIG. 6C, the G-rich sequence 110 is positioned within target polynucleotide molecule 108 on the non-template strand, for example DNA strand 104. Sacrificial oxidation of the G-rich sequence 110 on the non-template strand 104 will not interfere with the ability of the target polynucleotide molecule 108 to be transcribed. As illustrated, target polynucleotide molecule 108 includes exon regions 118 and 122 and intron region 120. As illustrated, G-rich sequence 110 is positioned within intron region 120 on the non-template strand 104; however the G-rich sequence 110 can be positioned any where along DNA region 102 on the template strand 104 in electrical communication with the target polynucleotide molecule 108. Alternatively, more than one reducing agent can be found along the DNA region 102 on the non-template strand 104.

In the presence of an oxidizing environment, the G-rich sequence 110 is sacrificially oxidized and can potentially form oxidized nucleotides. However, the presence of the oxidized nucleotides on the non-template strand 104 does not interfere with the ability of the RNA polymerase to transcribe an RNA molecule from the coding strand 106.

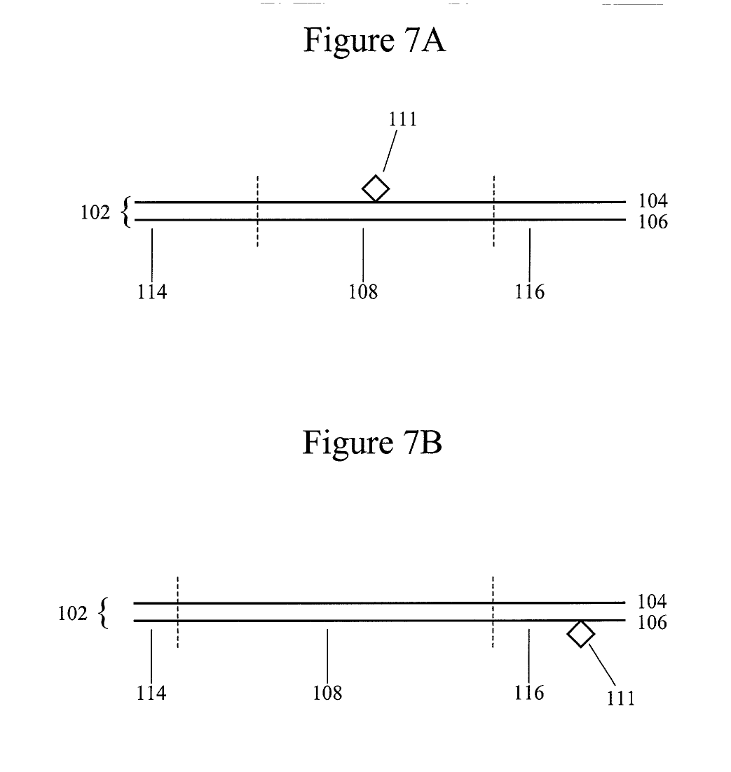

In another embodiment, as illustrated in FIG. 7A, the reducing agent is a reducing compound other than a G nucleotide or a G-rich nucleic acid segment, herein referred to as a “non-G reducing compound”. The non-G reducing compound 111 is coupled to the double-stranded DNA sequence 102. The non-G reducing compound 111 can be coupled to a nucleotide on the non-coding strand, for example DNA strand 104, at a position anywhere on the double-stranded DNA sequence 102 proximal to the target polynucleotide molecule. Coupling of the nucleotide to the non-G reducing compound 111 can be accomplished by various methods as described above. The presence of a nucleotide coupled to the non-G reducing compound 111, present on the non-coding strand 104, does not interfere with the transcription of the target polynucleotide molecule 108. Alternatively, as illustrated in FIG. 7B, the non-G reducing compound 111 can be coupled to the coding strand, for example DNA strand 106, at a position on the DNA sequence 102 outside, but proximal to, the transcribed region of the target polynucleotide molecule 108. The non-G reducing compound 111 can be inserted into the DNA sequence 102 through use of genetic engineering techniques. For example, an oligonucleotide containing a modified nucleotide (wherein the non-G reducing compound 111 is attached to the modified nucleotide) and restriction enzyme sites flanking the modified nucleotide can be synthesized, cut with the appropriate restriction enzymes, and cloned into the double-stranded DNA sequence 102 at a desired location. Alternatively, more than one non-G reducing compound 111 can be coupled to the DNA sequence 102.

Suitable non-G reducing compounds include, for example, oxidizable polycations. Examples of useful non-G reducing compounds include Vitamin K; tocopherol (Vitamin E); pyridoxine (Vitamin B₆) ; riboflavin (Vitamin B₂); 4,5-dihydro-4,5-dioxo-1H-pyrrolo-[2,3-f]quinoline-2,7,9-tricarboxylic acid (PQQ); 2,5-diaziridinyl-3,6-bis(carboethoxy-amino)-1,4-benzoquinone (AZQ); DOPA; dopamine; norepinephrine; adrenaline dihydroxyphenylglycolaldehyde; cobalamine (Vitamin B₁₂) and its derivatives such as 5′-deoxyadenosylcobalamine; and hydroxylated and deaminated tryptophan derivatives. Preferably, the non-G reducing compounds are small molecules having one or more phenolic OH functions in ortho or para positions and are maintained as phenols, diphenols or polyphenols.

The non-G reducing compound 111 can be coupled to the nucleotide or nucleotides of a nucleic acid sequence, for example, DNA sequence 102, through various types of chemical bonding including covalent, ionic, coordinative, van der Waals, or hydrogen bond (or combinations of these bonds). Typically, the non-G reducing compound 111 is coupled to the nucleotide(s) through covalent bonding. One useful method to couple the non-G reducing compound 111 to a nucleotide is through use of a spacer moiety. Spacer moieties can include, for example, alkyl spacers, aminoalkynyl spacers, aminoallyl spacers, and the like. The spacer moiety can be attached to a portion of the nucleotide, for example, from an atom on the base moiety of the nucleotide. Modified nucleotides containing reactive spacer moieties, for example 5-(3-amino allyl)deoxyuridine 5′-triphosphate can be obtained commercially (Molecular Probes, Eugene Oreg.), and can be incorporated into a DNA sequence. Other modified nucleotides containing a variety of other amine-reactive spacer moieties can also be obtained commercially (PerkinElmer Life Sciences Inc., Boston, Mass.). The non G-reducing compound 111, reactive with a portion of the spacer moiety, can then be coupled to a nucleotide(s) of a particular DNA sequence under the appropriate conditions.

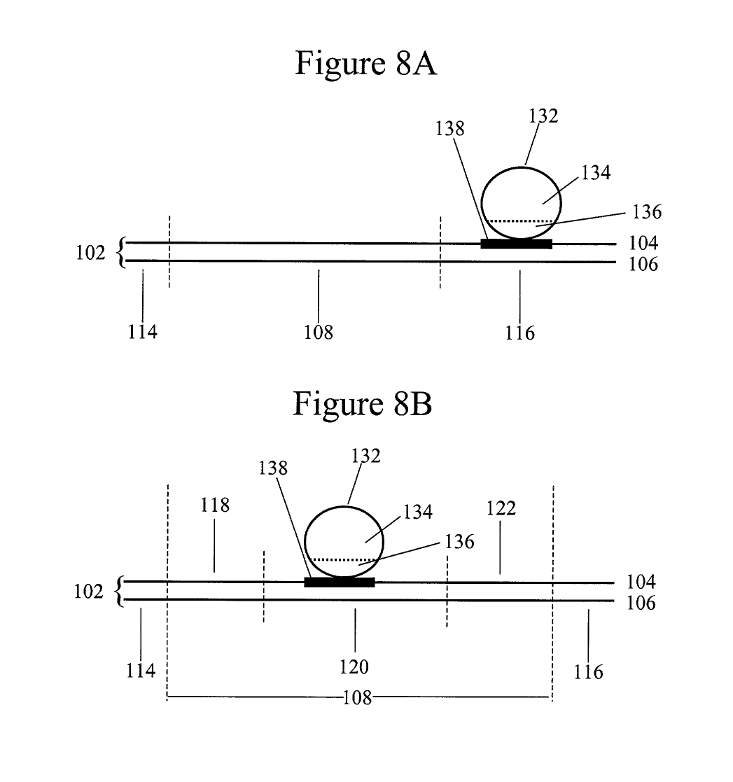

As illustrated in FIG. 8A, protection of the target polynucleotide molecule from oxidative damage can also be accomplished by positioning a reducing polypeptide 132 on a portion of the double-stranded DNA sequence 102. The polypeptide 132 is more readily reduced than the target polynucleotide molecule 108. Positioning of the reducing polypeptide 132 on a portion of the double-stranded DNA sequence 102 can be accomplished by providing a polypeptide 132 that includes at least a reducing portion 134 and a DNA binding portion 136. The reducing polypeptide 132 (also referred to as a “reducing chimeric polypeptide”) binds to a polypeptide-binding DNA sequence 138. The interaction of the DNA binding portion 136 and the protein-binding DNA sequence 138 typically occurs through hydrogen bond formation and van der Waals interactions between atoms of the polypeptide and the DNA.

The DNA binding portion 136 can include amino acid sequences of known DNA binding proteins. Likewise, the DNA sequence can include a polypeptide binding DNA sequence 138 that interacts with the DNA binding portion 136 of the reducing polypeptide 132. DNA binding proteins and DNA sequences that specifically bind these proteins are known, and include, for example, homeodomain protein:homeobox DNA sequence interations, zinc finger protein:DNA interactions, leucine-zipper protein:DNA interactions, helix-loop-helix protein:DNA interactions, and the like.

The reducing portion 134 of the reducing polypeptide 132 can include fragments or portions of proteins or polypeptides that are easily reduced, for example, thioredoxin, glutaredoxin, other members of the thioredoxin superfamily of thioldisulfide oxidoreductases, and polypeptides containing a relatively high cysteine content.

Polynucleotides encoding reducing polypeptides 132 and containing at least a DNA binding portion 136 and a reducing portion 134 can be constructed using genetic engineering techniques. Polynucleotides encoding reducing polypeptides 132 can be expressed in the presence of the target polynucleotide molecule 108 that is located proximal to the polypeptide-binding DNA sequence 138.

Alternatively, as illustrated in FIG. 8B, the protein-binding DNA sequence 138 can be located within the target polynucleotide molecule 108, preferably within an intron 120, for example. The protein binding DNA sequence 138 can either be on non-template strand 104 or template strand 106. The presence of a protein-binding sequence 138 in an intron, for example intron 120, will not affect the protein product encoded by target polynucleotide molecule 108, since the protein-binding DNA sequence 108 will be spliced out during pre-mRNA splicing.

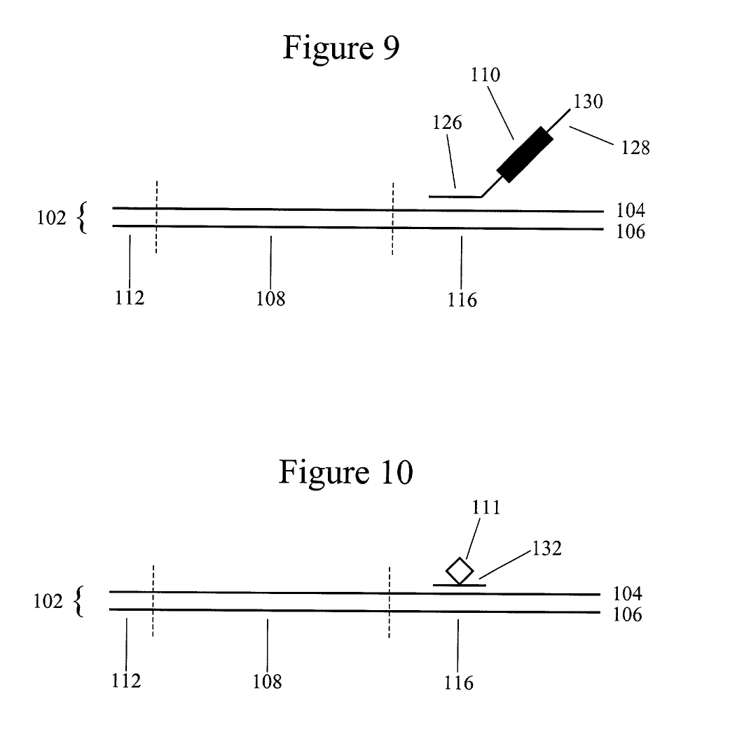

As illustrated in FIG. 9, the reducing agent 110 can be a G-rich nucleic acid sequence 110 that is positioned proximal to the target polynucleotide molecule 108 via a G-rich linker oligonucleotide 130. In this embodiment, a G-rich linker oligonucleotide 130 contains a first linker segment 126 and a second linker segment 128. The first linker segment 126 contains a nucleotide sequence that is complimentary to a sequence found on the double stranded DNA 102, for example, and as shown in FIG. 9, a sequence on non-coding strand 104 in second proximal nucleic acid region 116. This complimentary sequence allows the G-rich linker oligonucleotide 130 to hybridize to the target polynucleotide molecule 108 or a nucleic acid sequence proximal to the target polynucleotide molecule 108. The hybridization can be directed to any portion of the double stranded DNA 102 so that the G-rich linker oligonucleotide 130 is positioned proximal to the target polynucleotide molecule 108. The G-rich linker oligonucleotide 130 can be complimentary to either the first DNA strand 104 or the second DNA strand 106. Preferably, the complimentarity between the first linker segment 126 and the double stranded DNA 102 is over at least 8 base pairs, more preferably, at least 14 base pairs, and most preferably at least 20 base pairs.

The second linker segment 128 contains a G-rich sequence 110. The G-rich sequence 110 is preferably located within a distance from the target polynucleotide molecule 108 where, in the presence of an oxidizing agent and the formation of a hole in the target polynucleotide molecule 108, the hole is efficiently transmitted from the target polynucleotide molecule 108, along the linker oligonucleotide 124, and to the G-rich sequence 110, where the G-rich sequence 110 is sacrificially oxidized.

As illustrated in FIG. 10, the reducing agent can be a non-G reducing compound 111 that is coupled to a second linker oligonucleotide 132 and positioned proximal to the target polynucleotide molecule 108 by the hybridization of the linker oligonucleotide 132 to the DNA sequence 102. Preferably, the spacer moiety and the non-G reducing compound 111 to which it is attached do not interfere with the ability of the second linker oligonucleotide 132 to hybridize to a nucleic acid sequence proximal to the target polynucleotide molecule 108.

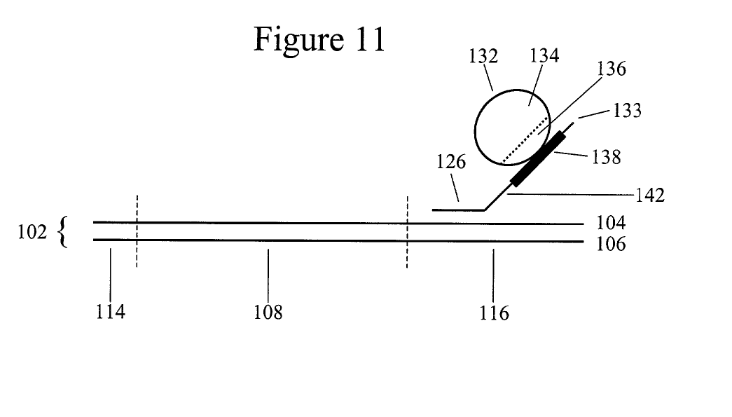

As illustrated in FIG. 11, a reducing polypeptide 132 including at least a reducing portion 134 and a DNA binding portion 136 can be positioned proximal to the target polynucleotide molecule 108 via a protein-binding linker oligonucleotide 133. The protein-binding linker oligonucleotide 133 contains a first linker segment 126 that is complimentary to a sequence found on the double stranded DNA 102 and a second linker segment 142 that contains a polypeptide-binding DNA sequence 138. The protein-binding linker oligonucleotide 133 can hybridize to the double stranded DNA sequence 102 at any position on or proximal to the target polynucleotide molecule 108 and to either the first DNA strand 104 or the second DNA strand 106.

In one useful application, linker oligonucleotides that are coupled to reducing agents can be used in PCR reaction as primers for the amplification of a target polynucleotide molecule 108. In this application, an upstream and a downstream linker oligonucleotide that hybridizes to nucleic acid sequences flanking the ends of the target polynucleotide molecule 108 are used as primers in a PCR reaction to amplify the target polynucleotide molecule 108. Either or both upstream or downstream primer can contain a reducing agent. The PCR products can therefore include a reducing agent on one end of the molecule or on both ends.

Cathodic protection in a PCR reaction may be desirable, since, in the early cycles of a PCR reaction oxidative damage to nucleotides could potentially create mutated products. This mutated sequence can then be propagated in subsequent PCR cycles. In an oxidative environment, protection of a target polynucleotide molecule 108 serving as template for a PCR reaction can reduce the probability that the target polynucleotide molecule 108 and its replicated products will contain mutations.

In other applications, for example DNA microarray technology, cathodic protection can also be useful. In the presence of an oxidative environment, target polynucleotide molecules 108 immobilized on microarrays can potentially become mutated. In some circumstances it is necessary or desirable to immobilize a relatively short, for example, about 12 nucleotide, target polynucleotide molecule on the microarry. Oxidative damage to one or more nucleotides or one or more of these relatively short target polynucleotide molecules on the array can potentially alter the hybridization properties of the target polynucleotide molecules with a sample being probed for the presence of nucleic acids. In this case, cathodic protection may be desirable to prevent nucleotide mutation of the target polynucleotide molecule.

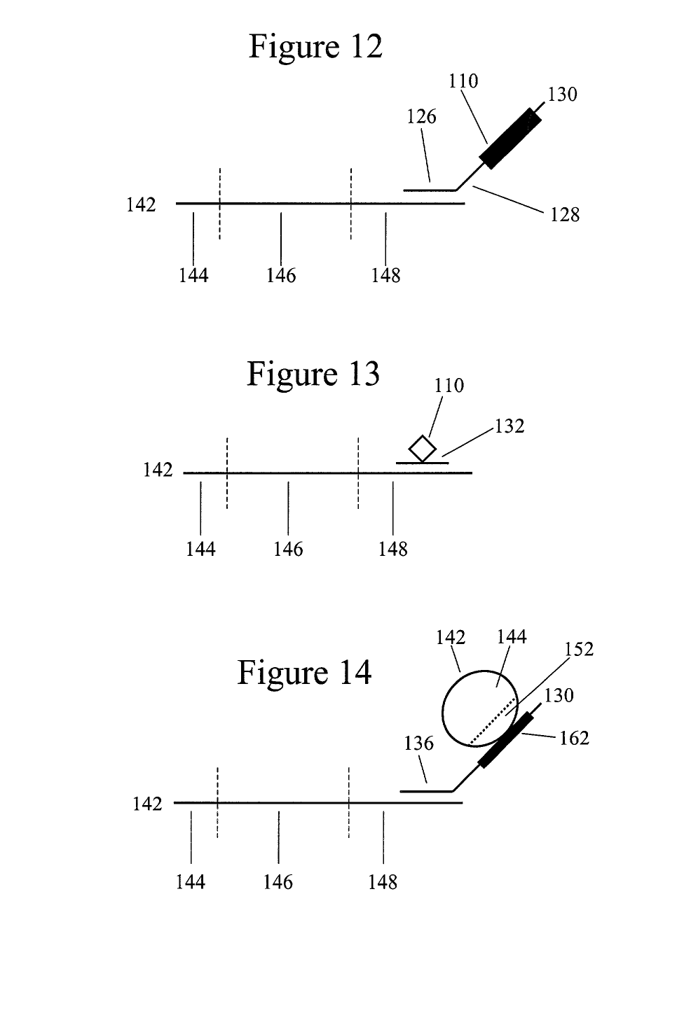

As illustrated in FIG. 12, the target polynucleotide molecule can be a RNA molecule that is protected by a hybridized G-rich linker oligonucleotide 130. As illustrated, the RNA molecule 142 contains a 5′ untranslated region 144, a translated region 146, and a 3′ untranslated region 148. Typically, a RNA molecule having these elements is a messenger RNA (mRNA) molecule. The G-rich linker oligonucleotide 130 contains a first linker segment 126 and a second linker segment 128. The first linker segment 126 includes a nucleotide sequence that is complimentary to a sequence found on the RNA molecule 142. This complimentary sequence allows the G-rich linker oligonucleotide 130 to hybridize to the RNA molecule 142. The hybridization is preferably directed to a portion of the RNA molecule 142 that does not interfere with the expression of the RNA molecule 142, for example, the translation or the binding of proteins involved in the expression of the protein product encoded by RNA molecule 142. Preferably, the complimentarily between the first linker segment 126 and the RNA molecule 142 is over at least 8 base pairs, more preferably, at least 14 base pairs, and most preferably at least 20 base pairs.

As illustrated in FIG. 13, an RNA molecule 142 can be protected by a hybridized second linker oligonucleotide 132. The second linker oligonucleotide 132 contains a reducing agent that is a non-G reducing compound 111 coupled to the second linker oligonucleotide 132.

As illustrated in FIG. 14, an RNA molecule 142 can be protected by a hybridized second linker oligonucleotide 132. The second linker oligonucleotide 132 contains a reducing agent that is a non-G reducing compound 111 coupled to the second linker oligonucleotide 132.

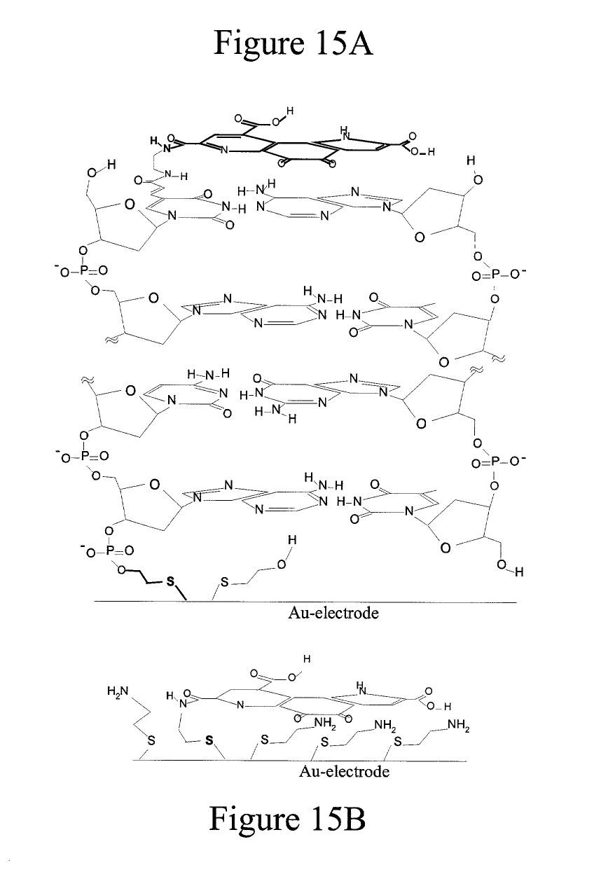

As illustrated in FIG. 15, the RNA molecule 142 can be protected by a reducing polypeptide 132 that contains at least a reducing portion 134 and a DNA binding portion 136 and is hybridized to the RNA molecule 142 via a protein-binding linker oligonucleotide. The protein-binding linker oligonucleotide 133 contains a first linker segment 126 that is complimentary to a sequence found on the RNA molecule 142 and a second linker segment 142 that includes a polypeptide-binding DNA sequence 138. The protein-binding linker oligonucleotide 133 can hybridize to the RNA molecule 142 preferably on a portion of the RNA molecule 142 that does not interfere with the expression of the RNA molecule.

This invention is can be particularly useful in cells or organisms where the DNA repair machinery has been compromised. DNA repair machinery can include genes and proteins responsible for the recognition, excision, and replacement of chemically damaged nucleotides, for example nucleotides that have been modified by oxidizing agents.

This invention can also be particularly useful in conditions where the target polynucleotide molecule 108 is to be expressed in the presence of ionizing radiation. The ionizing radiation in this case can lead to the formation of intracellular, extracellular oxidative compounds. For example, in an organism or cell culutre treated with an dose of ionizing radiation, for example gamma or X-ray electromagnetic radiation.

In an alternative embodiment, the reducing agent is not sacrificed, but is reversibly oxidized. The reducing, oxidizing centers of the invention are preferably at least 100 mV reducing versus the potential where the nucleic acid sequence to be protected is electrocatalytically oxidized, and preferably at least about 200 mV.

In an alternative method of the invention where increased mutation, chemo-sensitivity, and radiation sensitivity are desired, a target polynucleotide molecule may be stripped of G-rich protective areas. For example, a G-rich sequence positioned proximal to a target polynucleotide molecule can be mutated or deleted to remove the protective effect of the G-rich sequence. In vitro, removal of G-rich sequences can be accomplished, for example, by restriction digest and ligation or by using PCR techniques. In vivo, a G-rich sequence can be removed by, for example, transformation of a DNA sequence that results in the recombination and “knock out” of the G-rich sequence. Nucleic acid constructs, vectors, and the like containing a nucleic acid sequence where increased frequency of genetic mutation, chemo-sensitivity, or radiation sensitivity is desired, can be designed and synthesized to contain adjacent and proximal nucleic acids having few G residues, e.g., a mole fraction of G that is 0.25 or less.

The invention provides methods for modulating mutagenesis by altering the oxidation of nucleic acids. Increased exposure to oxidizing agents such as nitroso-compounds, quinones derived of polycyclic aromatic hydrocarbons and heterocyclic compounds, some recognized as carcinogens or mutagens, can deplete the reduction capacity of any G-rich protective regions normally associated with the nucleic acid sequence, for example, in CpG islands, resulting in an increased rate of mutation. Increased exposure to reducing agents, particularly reducing agents, such as G-rich protective nucleic acid segments of the invention positioned proximal to the protected nucleic acid sequences, or phenol-function comprising reducing agents provide a reduced rate of mutation.

According to the method of the invention, analysis of nucleic acid sequences in cells or tissues for the integrity of G-rich domains, for example, telomeres, can provide a measure of the cell's or tissue's susceptibility to mutation, for example, cancer causing mutations. Analysis for oxidized segments can be carried out by established means, for example by enzymatic hydrolysis followed by mass spectroscopy of the hydrolysate.

This specification contains numerous references to publications, each of which is hereby incorporated by reference for all purposes.

The present invention will now be illustrated by the following non-limiting examples.

### EXAMPLES

### Example 1

Gold Electrodes. Gold (99.99%) films of 100 nm thickness were sputtered on freshly cleaved and Ar plasma ion-etched muscovite mica faces, by the procedure of Golan, Y. et al. ((1992) Surf Sci, 264:312) and Ron, H. et al. ((1998) Langmuir, 14: 1116). Immediately after the sputtering the films were annealed at 290° C. for 2.5 hours and then allowed to cool over 1 hour to room temperature. The geometrical area of the electrodes was 1 cm². To strip the impurities adsorbed during the annealing step, the gold films were cleaned in 30% H₂O₂/70% H₂SO₄, rinsed with DI-water, then soaked in absolute EtOH for 20 minutes (to reductively strip any chemisorbed O₂), and immediately immersed in and reacted with the monolayer-forming disulfide solution.

Monolayer Formation. The Au—S—(CH₂)₂—NH₂ monolayers were formed by immersing the Au films in 0.02 M cystamine for at least 2 hours (Katz, E. et al., 1994, J Electroanal Chem, 367:59). The Au—S—(CH₂)₂—NH—PQQ monolayers were formed by incubating the Au—S—(CH₂)₂—NH₂ electrodes in a solution of 3×10⁻³ M PQQ, 10 mM EDC, and 10 mM sulfo-NHS in pH 7.2 HEPES. This procedure leads to preferential condensation of the 7-carboxylic acid function of PQQ with amines of the monolayer (Willner, I. et al., 1996, J Am Chem Soc, 118:10321).

The Au—S—(CH₂)₂—ss-oligo—NH—PQQ/Au—S—CH₂—CH₂—OH monolayer was formed as follows: 3′-ACGAAGGCTGAT-5′ oligonucleotide, mono-esterified with (HO—CH₂—CH₂—S—)₂ at the 3′-phosphate end and modified with a —CH₂—CH═CH—CO—NH—CH₂—CH₂—NH₂ function at the C-5 position of the 5′-thymine, was purchased from Genosys (The Woodlands, Tex.). The disulfides of the hybrid were next reacted with the gold surface by immersing the gold electrode for at least 24 hours in the solution containing all components of the hybridization solution except for the complementary oligonucleotide. Incubation with a solution of 3×10⁻³ M PQQ, 10 mM EDC, and 10 mM sulfo-NHS in pH 7.2 HEPES buffer lead to preferential condensation of the 7-carboxylic acid function of PQQ (Willner, I. et al., 1996, J Am Chem Soc, 118:10321) with the amine function at the 5′-thymine. Immobilization, other than by covalent attachment to the amine function at the 5′-thymine, did not take place. When the EDC coupling agent was omitted, the cyclic voltammograms did not show detectable PQQ peaks.

The double-stranded oligonucleotide monolayer, Au—S—(CH₂)₂—ds—oligo—NH—PQQ/Au—S—CH₂—CH₂—OH, was formed by a similar procedure, except that the 3′-ACGAAGGCTGAT-5′ oligonucleotide, mono-esterified with (HO—CH₂—CH₂—S—)₂ at the 3′-phosphate end and modified with a —CH₂—CH═CH—CO—NH—CH₂—CH₂—NH₂ function at the C-5 position of the 5′-thymine 0.2 mM solution, was hybridized with an equal volume 0.2 mM of its nonesterfied and unmodified complementary strand for 2 h at ambient temperature in the hybridization solution prior to reacting its disulfides with the gold surface. Incubation with a solution of 3×10⁻³ M PQQ, 10 mM EDC and 10 mM sulfo-NHS in pH 7.2 HEPES buffer led to preferential condensation of the 7-carboxylic acid function of PQQ with the amine function of the spacer chain on the terminal 5′-thymine. Immobilization of PQQ, other than by covalent attachment to the terminal thymine, did not occur. When the EDC coupling agent was omitted, the cyclic voltammograms did not show detectable PQQ peaks.

Two mismatched base pairs were introduced in the Au—S—(CH₂)₂—ds—oligo—NH—PQQ hybrids by hybridizing with the 3′-TGCTTAT-GACTA-5′ imperfectly complementary oligonucleotide instead of the perfectly complementary 3′-TGCTTCCGACTA-5′-oligonucleotide. The procedure was identical with that through which the Au—S—(CH₂)₂—ds—oligo—NH—PQQ/Au—S—CH₂—CH₂—OH monolayer was formed, except that the concentration of the oligonucleotide with two mismatched base pairs in the hybridization solution was 0.1 mM.

Experiments on Bulk DNA. Calf thymus DNA (cat. no. D3664) and soybean peroxidase (cat. no. P1432) were obtained from Sigma, St. Louis, Mo. Phosphate buffered test solution was made using Dulbecco's PBS (cat. no. 28374) from Pierce Chemical Co., Rockford, Ill. Poly(ethylene glycol) diglycidyl ether (PEGDGE) cross-linker (cat. no. 08211) was purchased from Polysciences, Warrington, Pa. All other reagents were purchased from Aldrich, Milwaukee, Wis.

The osmium-containing polymer was a copolymer of poly(acrylamide) and poly-(vinylimidazole) complexed with osmium 4,4′-dimethyl-2,2′-bipyridine (de Lumley, T. et al., 1995, Anal Chem, 67:1332). The experiments were carried out in PBS containing 1 mM H₂O₂, using a 3 mm diameter vitreous carbon electrode. The electrode was rotated at 1000 rpm and poised at SCE potential.

The component coating solutions of the SBP electrodes were prepared as follows. The SBP solution (20 mg/mL in 0.1 M NaHCO₃) was mixed with an equal volume of a NaIO₄ (12 mg/mL in water) solution. The mixture was kept in the dark for 2 hours, then centrifuged and washed with a copious volume of 0.1 M NaHCO₃ using a Microcon 30 concentrator. Calf thymus DNA (50 μl at 1.2 mg/mL in water was mixed with 1 mL of methyl imidazole buffer (20 mM, pH7) containing 0.15 M EDC and 2.5 mM hydrazine monohydrate and reacted overnight (18 hours) at room temperature. The treated DNA was then washed with methyl imidazole buffer in a Micron concentrator. Electrodes were prepared by depositing 6 μl of 85 wt % DNA (2.93 mg/mL) or PAA—PVI—Os—Hz (5 mg/mL), 5 wt % PEGDGE (0.5 mg/mL) and 10 wt % SBP (9 mg/mL) onto the 3 mm glassy carbon electrodes. The electrodes were cured in a humid atmosphere for 16 hours and then overnight in ambient air before testing. Prior to testing the electrodes were washed in PBS for 15 minutes.

Instrumentation. The CVs were measured with a computer-controlled low-noise bipotentiostat (CH Instruments, model 832) at ambient temperature in a standard three-electrode cell, with a coiled plantinum wire auxiliary electrode and a double junction Luggin capillary Ag/AgCl (saturated KCl internal solution) or an SCE reference electrode. The electrolyte was pH 7.2 HEPES buffer with 0.7 M TEATFB. The modified electrodes wre rinsed with D1 water before the measurements. X-ray photoelectron spectra (XPS) were obtained with a Physical Electronics Phi model 5700 ESCA system, operated at 10⁻¹⁰ Torr, using monochromatic Al Kα X-rays at 1486.6 eV at 300 W with 2 mm focused filament. Its hemispherical analyzer was operated at a band-pass energy of 93.9 eV (survey scan, time/step=0.2 s at 0.4 eV/step) or 11.75 eV (high-resolution scan, time/step=1 s at 0.1 eV/step) with the entrance aperture (4 μm) at 45° relative to the sample surface.

FIG. 15A illustrates the 12 base PQQ-bound double-stranded oligonucleotide (3′-ACGAAGGCTGAT-5′) hybrid on gold, Au—S—(CH₂)₂-double stranded oligo—NH—PQQ. The PQQ redox function is attached to 5′thymine via a C-5—CH₂—CH═CH—C)—NHCH₂—CH₂—NH₂ spacer arm. The length of the unit is about 49±2 Å.

FIG. 15B is a schematic drawing of Au—S—(CH₂)₂—NH—PQQ, having no nucleotide attached. The remaining available gold surface is occupied by cystamine.⁶

### Example 2

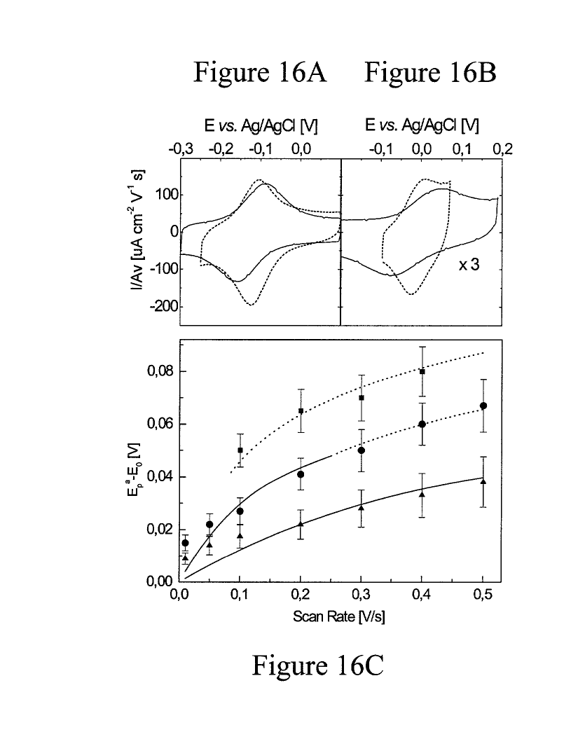

FIGS. 16A and 16B are graphic representations of cyclic voltammograms, normalized for the scan rate, for the electrooxidation/ reduction of PQQ/PQQ²⁻ functions bound to the termini of monolayers on gold. Dotted lines show scan rates of 10 mVolts per second; solid lines show scan rates of 500 mVolts per second. Electrooxidation/reduction of Au—S—(CH₂)₂—NH—PQQ is shown in FIG. 16A and of Au—S—(CH₂)₂-double stranded oligo—NH—PQQ is shown in FIG. 16B.

FIG. 16C is a graph showing the dependence of the peak separation Eₚ^{a}-E_{O} on the scan rate for monolayers on gold. Data for monolayers of Au—S—(CH₂)₂—NH—PQQ are shown as triangles; of the 12 base pair are shown as circles; and of the Au—S(CH₂)₂-double stranded oligo—NH—PQQ having a 2 base pair mismatch are shown as squares. Data are fit to the two domains of the theoretical model of Laviron, 1979, J Electroanal Chem, 101:19-28, and are shown as dashed and solid lines as described in detail in Hartwich, G. et al, 1999, J Am Chem Soc, 121:10803-10812).

The above examples are meant as illustrative of the invention, and not to be limiting of the invention as claimed below.

We claim:

1. A method for protecting a target polynucleotide molecule from oxidative damage, the method comprising positioning at least one reducing agent proximal to the target polynucleotide molecule.

2. The method of claim 1, wherein the reducing agent is positioned on a non-template strand.

3. The method of claim 1, wherein the target polynucleotide molecule is a DNA molecule having a protein coding region, and the reducing agent is positioned outside the coding region.

4. The method of claim 3, wherein the DNA is expressed in cultured cells.

5. The method of claim 1, wherein the reducing agent comprises a G-rich nucleic acid segment.

6. The method of claim 5, wherein the G-rich nucleic acid segment contains a mole fraction of guanine about 0.4 to about 0.5.

7. The method of claim 1, wherein the reducing agent is a non-G reducing compound.

8. The method of claim 7, wherein positioning comprises covalently coupling the non-G reducing compound proximal to the target polynucleotide molecule via a spacer moiety.

9. The method of claim 7, wherein the non-G reducing compound is vitamin K, tocopherol (Vitamin E), pyridoxine (Vitamin B₆), riboflavin (Vitamin B₂), PQQ, DOPA, dopamine, norepinephrine, adrenaline dihydroxyphenylglycolaldehyde, cobalamine (Vitamin B₁₂), 5′-deoxyadenosylcobalamine, hydroxylated tryptophan, deaminated hydroxylated tryptophan, or a combination thereof.

10. The method of claim 1, wherein the reducing agent is a reducing polypeptide containing a DNA binding site and wherein the reducing polypeptide is bound to a protein-binding DNA sequence positioned proximal to the target polynucleotide molecule.

11. The method of claim 1, wherein positioning comprises coupling the reducing agent proximal to the target polynucleotide molecule via a linker oligonucleotide.

12. The method of claim 11, wherein the linker oligonucleotide comprises:
a first linker segment comprising a first nucleic acid sequence capable of hybridizing with a second nucleic acid sequence that is located proximal to the target polynucleotide molecule, and
a second linker segment comprising a guanine-rich nucleic acid sequence.

13. The method of claim 11, wherein the linker oligonucleotide comprises:
a first linker segment comprising a first nucleic acid sequence that hybridizes with a second nucleic acid sequence that is located proximal to the target polynucleotide molecule; and
a second linker segment coupled to a non-G reducing compound.

14. The method of claim 11, wherein the linker oligonucleotide comprises:
a first linker segment comprising a first nucleic acid sequence that hybridizes with a second nucleic acid sequence that is located proximal to the target polynucleotide molecule; and
a second linker segment comprising a protein-binding DNA sequence; wherein the reducing polypeptide comprising a DNA binding site is bound to the protein-binding DNA sequence.

15. The method of claim 11, wherein the linker oligonucleotide is amplified in a PCR reaction.

16. The method of claim 1, wherein the target polynucleotide molecule comprises an RNA molecule.

17. A vector comprising a target polynucleotide molecule and a reducing agent positioned proximal to the target polynucleotide molecule.

18. A vector comprising a target polynucleotide molecule, a reducing protein binding site, and a nucleic acid seqence encoding a reducing protein, wherein the reducing protein binds the reducing protein binding site.
